(19) United States
(12) Patent Application Publication
(10) Pub. No.: US 2010/0168866 A1
(43) Pub. Date: Jul. 1, 2010

(54) Femoral head prosthesis assembly and operation instruments thereof

(76) Inventor: Grant Lu-Sun Shih, Xindian City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/458,572

(22) Filed: Jul. 16, 2009

(30) Foreign Application Priority Data
Dec. 26, 2008 (TW) 097151105

(51) Int. Cl. A61F 2/36

(52) U.S. Cl. 623/23.14; 623/23.12

(57) ABSTRACT

A femoral head prosthesis assembly and operation instruments thereof. The operation instruments include a hollow femoral neck holder with a configuration adapted to the surface configuration of the femoral neck and a shaper blade for cutting a replacement end of the femur into a predetermined configuration. A guide tube is disposed on a top face of the femoral neck holder. A transverse slot is formed on the femoral neck holder for indicating a femoral head cutting line. The femoral head prosthesis assembly includes a cap body and an artificial femoral head. A root section of the cap body has an inner surface in conformity to the surface of the femoral neck. A top section of the cap body has a cross section adapted to that of the replacement end. The cap body can be precisely securely bonded with the replacement end of the femur.

### BACKGROUND OF THE INVENTION

The present invention relates to a femoral head prosthesis assembly and operation instruments thereof. With the operation instruments, an operator can more precisely perform femoral head replacement and more securely bond the artificial femoral head with the natural femoral neck to promote operation quality.

It is known that a stem-type femoral prosthesis or a stemless femoral prosthesis is used in a traditional femoral head replacement. As shown in FIG. 1, the necrotic or worn femoral head 42 along with the femoral neck 41 is generally directly cut off from the femur 4 along an oblique cutting line L4 marked on the root of the femoral neck 41. Then the femoral medulla is extracted to ream the femoral medullary cavity. Thereafter, an artificial femoral stem is implanted in the femoral medullary cavity and an artificial femoral head 5 is connected to the stem. Such operation is disclosed in U.S. Pat. No. 5,163,961, entitled COMPRESSION-FIT HIP PROSTHESIS AND PROCEDURE FOR IMPLANTATION THEREOF. Also, Chinese Utility Model Patent Publication No. CN2430959Y discloses a self-locked stemless hip prosthesis. According to this patent, the necrotic or worn femoral head 42 is cut off from the femoral neck and a cap body 51 is fixed on the remaining femoral neck with bolts. In practice, the above operations have some defects as follows:
1. With respect to traditional hemiarthroplasty in which a stem-type femoral prosthesis is used, it is necessary to cut off an extensive part of the femoral head and extract a large amount of femoral medulla for hemiarthroplasty. Therefore, the extent of trauma, the operation time and the blood loss are all greatly increased. This significantly increases operation risk as a whole.
2. It is hard to precisely control the dimension of the part of the femur, (such as the femoral neck), that needs to be cut off or extracted. Therefore, it is difficult to precisely match the surface of the stem-type femoral prosthesis or the inner surface of the cap body of the stemless femoral prosthesis with the surface of the remaining part of the femur. As a result, the femoral prosthesis can hardly securely bond with the rigid remaining part or the femoral neck of the femur. Moreover, stress concentration is likely to take place to directly and seriously affect operation result and recovery quality.
3. Every femoral prosthesis has a lifetime. Therefore, after a long period of use, the femoral prosthesis tends to wear, loosen or sink and may need revision. With respect to traditional hemiarthroplasty in which a stem-type femoral prosthesis is used, an extensive part of the femoral head is cut off in the primary operation so that the extent of trauma is pretty great. Consequently, in the revision, it is generally necessary to re-ream the femoral medullary cavity. Such operation inevitably increases the extent of trauma and is more difficult to perform.
4. With respect to traditional hemiarthroplasty in which a stemless femoral prosthesis is used, there is no scientific and precise measuring method or program for the part to be cut off. Therefore, the cutting position of the femoral neck is often decided simply by experience. Accordingly, the operation result apparently varies with the operators and it is hard to ensure good operation quality. Moreover, it is impossible to perform pre-operational evolution to simulate and evaluate the operation. Therefore, it is hard to give concrete pre-operation metal construction to a patient.
5. As shown in FIG. 1, the cap body 51 of the stemless femoral prosthesis is obliquely positioned on the cutting plane taken along the cutting line L4. In this case, it cannot be sure that the surface of the femoral neck 41 continuously tightly fits with the inner surface of the cap body 51. Consequently, the stress applied to the hip joint can be hardly uniformly distributed and transmitted and stress concentration is likely to take place in practical use. This affects durability of the stemless femoral prosthesis and needs improvement.

### SUMMARY OF THE INVENTION

It is therefore a primary object of the present invention to provide a femoral head prosthesis assembly, which can be more precisely assembled and implanted to improve fitting effect for hip joint replacement and enhance operation quality.

It is a further object of the present invention to provide the above femoral head prosthesis assembly, which is directly fixedly assembled with the femoral neck by a maximum area. A major part of the femoral neck is reserved without reaming the femoral medullary cavity. Accordingly, in case a revision is needed, a traditional hemiarthroplasty can be further performed to cut off the femoral neck and implant a stem-type femoral prosthesis in the femur. Therefore, the lifetime of the femur can be prolonged.

It is still a further object of the present invention to provide the above femoral head prosthesis assembly, which can be tightly fitted on the femoral neck and fully attached to the surface of both proximal shaped end with cancellous bone and distal inter-trochantor end with cortical bone thereof. Moreover, the femoral head prosthesis assembly extends to the distal end, that is, the root portion of the femoral neck to form a tapered inner cap with the shaped proximal end thereof. The femoral head prosthesis assembly contacts the surface of the remaining rigid root portion of the femoral neck by a maximum area so that the femoral head prosthesis assembly can bond with the remaining part of the femoral neck by enhanced strength. Accordingly, the femoral head prosthesis assembly can be securely bonded with the natural femoral neck with longer lifetime.

It is still a further object of the present invention to provide an operation instrument for femoral head replacement. The operation instrument is precisely made on the basis of data achieved by means of scanning the surface of the femoral neck of a patient with a medical volume scanning equipment. The operation instrument is manufactured in accordance with biological dynamics to reduce cutting area of the femur and thus lower trauma caused by the operation. Moreover, with cooperative simulation software, the data can be input to a computer for an operator to perform a pre-operational evolution to simulate the operation and get experienced with various situations in the replacement procedure. Accordingly, the success possibility of the operation can be enhanced to have a patient have concrete confidence before the operation and thus lower operation risk.

According to the above objects, the femoral head prosthesis assembly of the present invention includes a cap body and an artificial femoral head. The cap body has a caved portion and a connection portion, which extend in opposite directions. The caved portion has a root section and a top section. The root section has an inner surface with a configuration and a dimension achieved by means of scanning the surface of the femoral neck of a patient with a medical volume scanning equipment. Those points of the periphery of the femoral neck that are spaced from a central line of the femoral neck by shortest distances are taken to compose a cutting loop defining a narrowest cross section. The top section has an inner surface with a configuration and a dimension adapted to those of the narrowest cross section. Accordingly, the top section can be tightly fitted on a protrusion portion formed on the femoral neck by cutting the femoral neck and the inner surface of the cap body can be tightly bonded with and located on the surface of the femoral neck. The artificial femoral head has a socket for fixedly fitting on the connection portion of the cap body.

The operation instruments for femoral head replacement of the present invention include a femoral neck holder and a shaper blade. The femoral neck holder is a hollow casing with a configuration adapted to the surface configuration of the femoral neck. The femoral neck holder is sectioned at a middle portion thereof to form a side opening. A guide tube is disposed on a top face of the femoral neck holder. The guide tube extends along the central line of the femoral neck for forming a central hole on the femoral neck. A transverse slot is formed on the middle portion of the femoral neck holder for indicating a cutting position. The shaper blade has a sleeve portion and a cutting blade portion. The cutting blade portion has a lower opening. A periphery of the opening is formed with a sharp blade section. The blade section has a dimension and a sectional configuration in conformity to those of a cutting loop composed of points of the periphery of the femoral neck, which points are spaced from a central line of the femoral neck by shortest distances. A guide member is disposed in a central hole of the femoral neck. The sleeve portion is fitted around the guide member for reciprocally cutting the proximal end of femoral neck into a predetermined configuration.

The present invention can be best understood through the following description and accompanying drawings wherein:

### BRIEF DESCRIPTION OF THE DRAWINGS

FIG. 1 is a view showing that a conventional artificial femoral head is implanted in a femur, also showing the most often seen femoral head cutting line in the traditional hip arthroplasty;

FIG. 2 is a view showing the primary cutting line of the femoral head and other relevant locating lines according to the present invention;

FIG. 3 is a view showing the cutting loop and replacement loop of the femoral neck according to the present invention;

FIG. 4 is a perspective view of the femoral neck holder of the present invention;

FIG. 5 is a top view of the femur after the femoral head is cut off along the primary cutting line according to the present invention;

FIG. 6 is a perspective view of the femur after the femoral head is cut off along the primary cutting line according to the present invention;

FIG. 7 is a view showing that the femoral neck holder of the present invention is capped on the end section of the femur after primary cutting;

FIG. 8 is a perspective view of the shaper blade of the present invention;

FIG. 9 is a sectional view of the shaper blade of the present invention;

FIG. 10 shows the operation of the shaper blade of the present invention;

FIG. 11 is a view showing a conic protrusion portion is formed on the femoral neck after cut with the shaper blade;

FIG. 12 is a perspective exploded view of the artificial femoral head assembly of the present invention;

FIG. 13 is a perspective assembled view of the artificial femoral head assembly of the present invention;

FIG. 14 is a side sectional assembled view of the artificial femoral head assembly of the present invention;

FIG. 15 is a perspective assembled view of the artificial femoral head assembly of the present invention as seen from another side;

FIG. 16 shows that the artificial femoral head of the present invention is housed in a cup implanted in an acetabulum; and

FIG. 17 is a perspective view of another embodiment of the artificial femoral head assembly of the present invention.

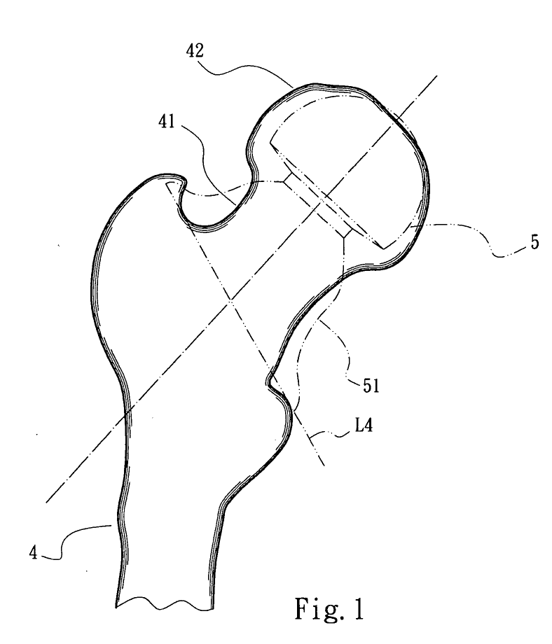

### DETAILED DESCRIPTION OF THE PREFERRED EMBODIMENTS

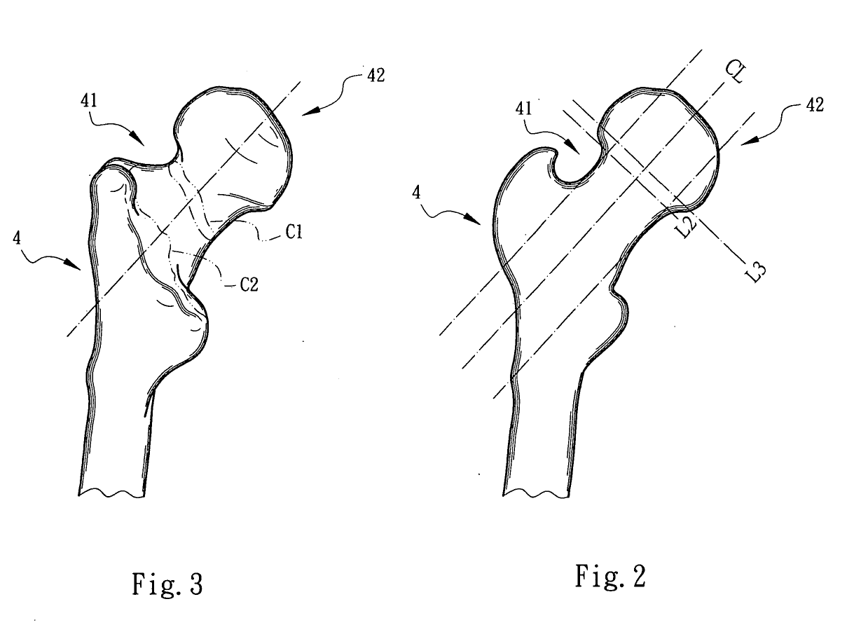

Please refer to FIGS. 2 and 3. Before the artificial femoral head replacement, as seen from one side, a femoral head-neck interface line L2 is properly taken at the junction between the femoral head 42 and femoral neck 41 of the femur 4. In addition, a primary cutting line L3 is taken in parallel to the femoral head-neck interface line L2. The primary cutting line L3 is spaced from the femoral head-neck interface line L2 in a direction to the femoral head 42 by a certain distance. A common central line CL of the femoral head 42 and femoral neck 41 is determined on the basis of the femoral head-neck interface line L2 or the primary cutting line L3. Also, the data of surface configuration of the femoral neck 41 are measured by means of a medical volume (three-dimensional) scanning equipment such as a computer tomography (CT) or a magnetic resonance imaging (MRI) equipment. Then, the shortest distances from the central line CL to the periphery of the femoral neck 41 are taken to obtain a cutting loop C1 defining a narrowest cross section. In addition, a maximum junction cross section is taken below the cutting loop C1 between the root of the femoral neck 41 and the femur 4. The periphery of the cross section forms a replacement loop C2.

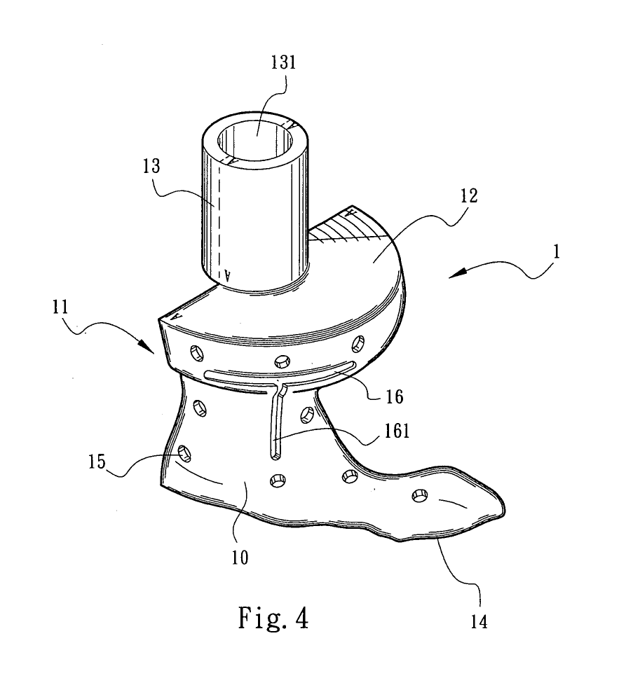

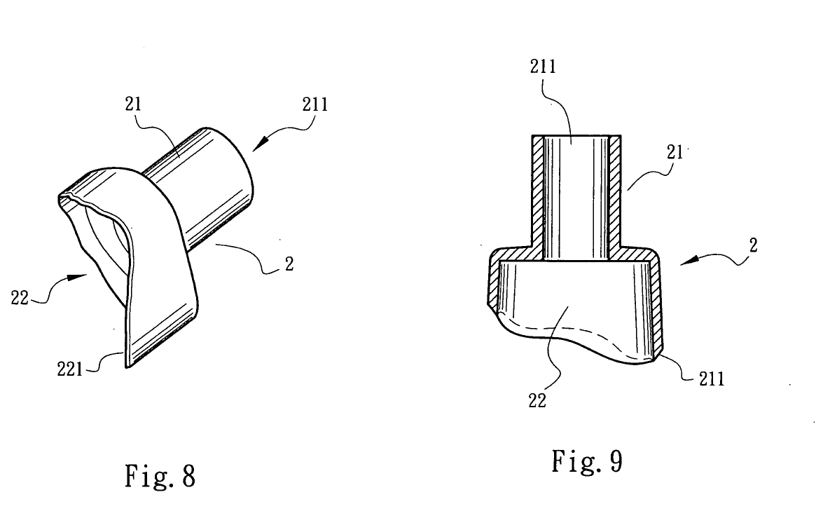

Referring to FIGS. 4 to 11, the operation instruments of the present invention mainly include a femoral neck holder 1 and a shaper blade 2. The femoral neck holder 1 essentially has a configuration, which is adapted to the position of the primary cutting line L3 in conformity to the surface configuration of the periphery of the femoral neck 41. The femoral neck holder 1 has a bottom portion 10 positioned under the cutting loop C1. The bottom portion 10 has an inner surface with a configuration and a dimension in conformity to or approximately in conformity to those of the outer surface of the femoral neck 41. In addition, the femoral neck holder 1 has a top plane face 12 corresponding to the primary cutting line L3. The top plane face 12 has a dimension not less than that of a cross section taken along the primary cutting line L3. The femoral neck holder 1 is longitudinally sectioned at a middle portion thereof to form a side opening 11. A guide tube 13 is disposed on the top plane face 12. The guide tube 13 has a guide hole 131 extending along the central line CL. A transverse slot 16 is formed on the middle portion of the femoral neck holder 1 beside the top plane face 12 in alignment with the femoral head-neck interface line L2. In addition, a longitudinal slot 161 is formed on the femoral neck holder 1 and extends from a middle point of the transverse slot 16 in parallel to the central line CL. Multiple locating holes 15 are further distributed over the femoral neck holder 1. A lower periphery 14 of the bottom portion 10 of the femoral neck holder 1 has a configuration adapted to that of the replacement loop C2 (as shown in FIG. 4). The shaper blade 2 is composed of a sleeve portion 21 and a cutting blade portion 22 connected with the sleeve portion 21. The cutting blade portion 22 is a hollow column with a straight periphery in parallel with the central line of femoral neck. The cutting blade portion 22 has a periphery formed with a sharp blade section 221. The blade section 221 has a configuration and a dimension in conformity to those of the cutting loop C1 (as shown in FIGS. 8 and 9).

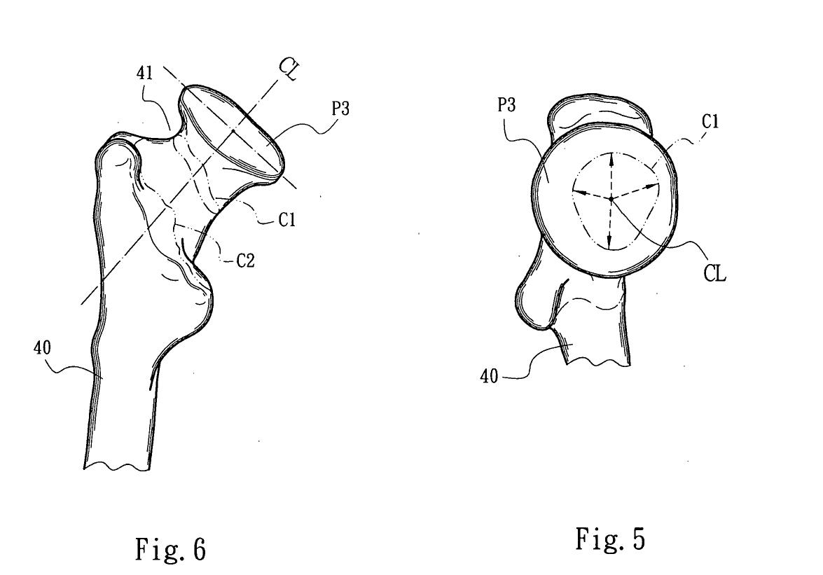

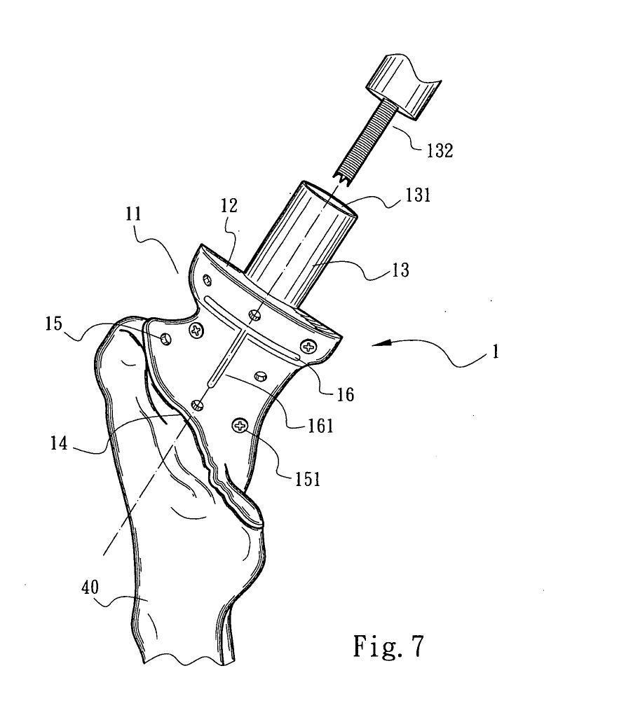

In operation, the femoral head 42 is first cut along the primary cutting line L3 to form a primary cutting face P3 on the femur 40 (as shown in FIGS. 5 and 6). The femoral neck holder 1 is capped on the femoral neck 41 through the side opening 11. Multiple temporary retainer members 151 are passed through the locating holes 15 to penetrate into the femur 40 so as to firmly fix the femoral neck holder 1 on the peripheral portion of the femoral neck 41 (as shown in FIG. 7). Thereafter, with the indication of the transverse slot 16, an operator can be sure of the true position of the femoral head-neck interface line L2. At the same time, the position of the central line L2 can be marked by means of the longitudinal slot 161. A drilling instrument 132 is extended into the guide hole 131 of the guide tube 13 to drill a central hole 43 on the primary cutting face P3 along the central line CL. Then, through the transverse slot 16, the femoral head is cut off along the femoral head-neck interface line L2.

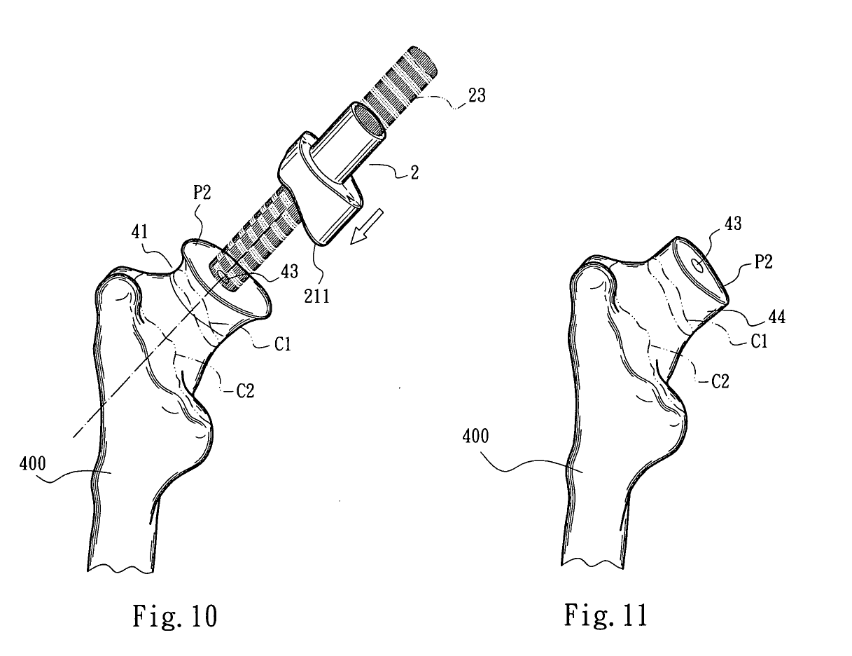

Thereafter, a guide member 23, (which can be a bar body), is inserted and locked in the central hole 43. The sleeve portion 21 of the shaper blade 2 is fitted around an outward extending portion of the guide member 23. The shaper blade 2 can be reciprocally moved along the guide member 23 (as shown in FIG. 10), whereby the blade section 221 of the cutting blade portion 22 can cut and mill off the periphery of the femoral neck 41 above the cutting loop C1 to form a substantially conic protrusion portion 44 with a central hole 43. The periphery of the protrusion portion 44 is in parallel with the central line of the femoral neck. The protrusion portion 44 has a profile approximately identical to that of the narrowest cross section of the femoral neck (as shown in FIG. 11).

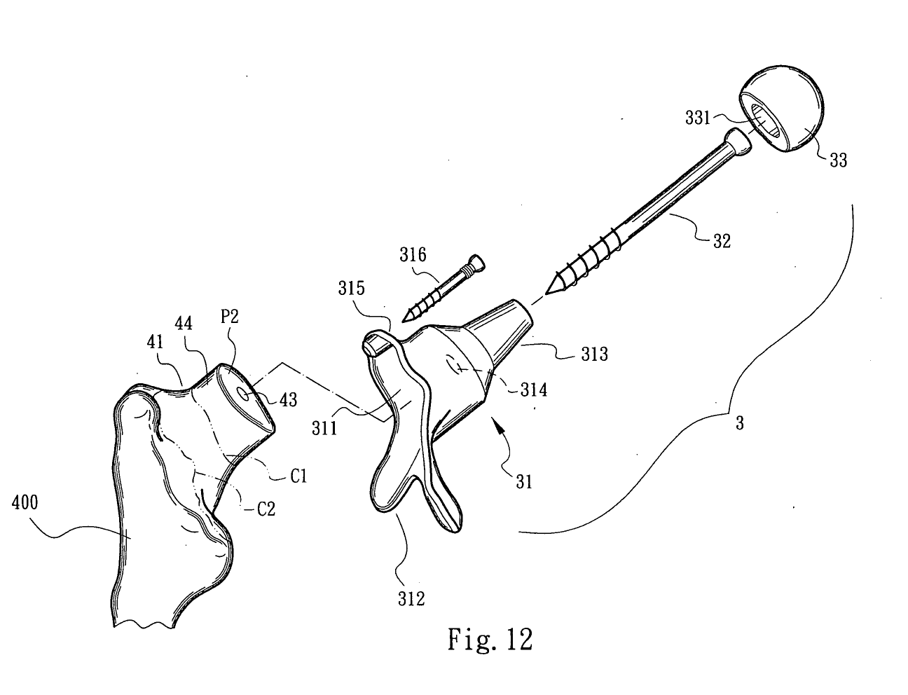

Referring to FIGS. 12 to 15, the femoral head prosthesis assembly, (that is, the artificial femoral head assembly 3), of the present invention includes a cap body 31 and an artificial femoral head 33. The cap body 31 has a caved portion 311 and a connection portion 313, which extend in opposite directions and communicate with each other through a through hole 314. The surface configuration data between the cutting loop C1 and the replacement loop C2 are taken from the surface configuration data of the femoral neck 41 as the data of the inner surface configuration of a root section 3111 of the caved portion 311. The caved portion 311 further has a top section 3112 above the root portion 3111. The configuration and size of the inner surface of the top portion 3112 at least necessarily adapt to or slightly smaller than those of the protrusion portion 44, which is in parallel with the central line of femoral neck beginning from the cutting loop C1 to the femoral head-neck interface line L2 and has a profile approximately identical to that of the narrowest cross section of the femoral neck. A lower periphery 312 of the cap body 31 has a configuration in conformity to that of the replacement loop C2. In addition, at least one locating hole 315 is formed on one side of the caved portion 311 as necessary (as shown in FIG. 12).

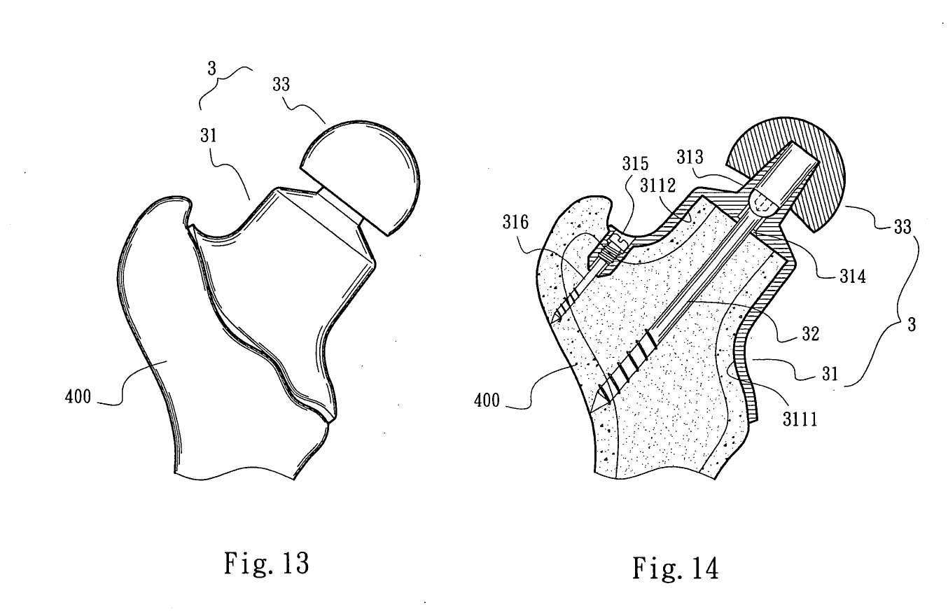

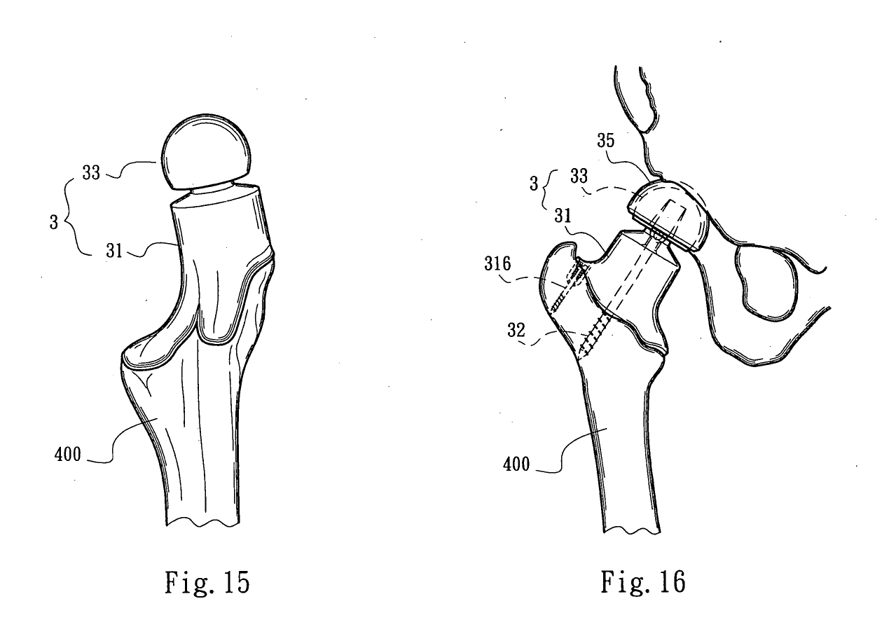

When assembled, the cap body 31 is capped onto a replacement end of the femur 400 with the protrusion portion 44. At this time, the protrusion portion 44 is snugly fitted into the top section 3112 of the cap body 31 with the root section 3111 snugly or slightly pressably attaching to the surface of the femoral neck between the cutting loop C1 and the replacement loop C2 (as shown in FIGS. 13 and 14). Under such circumstance, the cap body 31 can be highly snugly attached to and securely fitted on the femoral neck. Then, a locating member 32 (screw) is passed through the through hole 314 of the cap body 31 and extended (screwed) into the central hole 43 of the protrusion portion 44. In addition, an auxiliary locating member 316 is passed through the locating hole 315 and extended (screwed) into the femur 400 beside the protrusion portion 44. Accordingly, the cap body 31 is firmly located at the end section of the femur 400. Thereafter, an artificial femoral head 33 with a socket 331 is fitted onto the connection portion 313 of the cap body 31 and fixed thereon (as shown in FIG. 15) to complete the femoral head replacement. In practice, the artificial femoral head 33 can be relatively rotatably housed in a cup 35. In this case, the artificial femoral head assembly 3 and the cup 35 can be respectively implanted in the femur and the acetabulum to replace the necrotic hip joint of a patient.

The femoral neck holder 1 and the shaper blade 2 of the present invention help an operator to precisely cut, drill and mill the respective parts of the femur so as to enhance assembly precision and improve hip joint revision effect after operation as well as promote operation quality. Moreover, cooperative computer simulation software serves to provide pre-operational evolution for an operator. Accordingly, the operator can previously simulate the operation before it is really performed. In this case, the operator can get experienced with the operation to lower the possibility of error in the operation and ensure success of the operation. Furthermore, the cap body 31 is tightly fitted on the protrusion portion 44 of the top end of the femoral neck 41 and secured with the locating member 32 and auxiliary locating member 316. Therefore, the cap body 31 is more firmly bonded with the femoral neck 41 with better connection strength. The cap body 31 contacts the end section of the femur 400 by maximum effective replacement area. Therefore, the action force transmitted from the artificial femoral head assembly 3 to the femoral neck 41 can be distributed to respective parts of the end section of the femur 400 so as to avoid possible fracture due to stress concentration. Accordingly, the ability of the end section of the femur 400 to bear the loading force is greatly enhanced and the lifetime of the entire artificial femoral head assembly 3 can be prolonged. In the traditional operation, the femoral head 42 is directly cut off-along the root section of the femoral neck 41. In comparison with the traditional operation, according to the present invention, the femoral head 42 is cut off along the femoral head-neck interface line L2 above the cutting loop C1, while the major part of the femoral neck 41 below the cutting loop C1 is reserved. Therefore, in case the cap body 31 or any other component of the artificial femoral head assembly 3 is damaged and a revision is needed, a traditional operation can be further performed (to cut off the femoral neck 41 along the cutting line L4). Accordingly, the lifetime of the femur 400 can be prolonged.

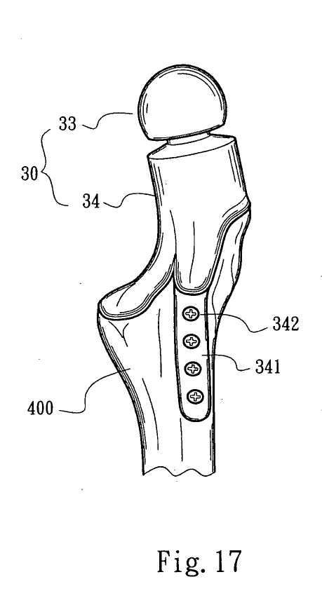

FIG. 17 shows another embodiment of the artificial femoral head assembly 30 of the present invention. In this embodiment, the artificial femoral head assembly 30 includes the same artificial femoral head 33 and another type of cap body 34. The cap body 34 has a structure based on the cap body 31. The cap body 34 is different from the cap body 31 only in that the cap body 34 additionally has a plate-like fixing extension portion 341 extending from the lower periphery of the cap body 34. Multiple locating members 342 (bolts) can be passed through the fixing extension portion 341 to penetrate (screw) into the femur 400. The fixing extension portion 341 serves as a reinforcing structure, which provides enhanced locating effect for the cap body 34, especially for a patient of osteoporosis.

In conclusion, with the femoral head prosthesis assembly and the operation instruments of the present invention, the operation quality is promoted, the lifetime of the artificial femoral head is prolonged and the operation risk is reduced. The above embodiments are only used to illustrate the present invention, not intended to limit the scope thereof. Many modifications of the above embodiments can be made without departing from the spirit of the present invention.

What is claimed is:

1. A femoral head prosthesis assembly comprising:
a cap body having a caved portion and a connection portion, which extend in opposite directions, the caved portion having a root section and a top section, the root section having an inner surface with a configuration and a dimension achieved by means of scanning the surface of the femoral neck of a patient with a medical volume scanning equipment, points of the periphery of the femoral neck, which are spaced from a central line of the femoral neck by smaller than or equal to the shortest distances being taken to compose a cutting loop defining a narrowest cross section, the top section having an inner surface with a configuration and a dimension adapted to those of the narrowest cross section, whereby the top section can be tightly fitted on a protrusion portion formed on the femoral neck by cutting the femoral neck and the inner surface of the cap body can be tightly bonded with and located on the surface of the femoral neck; and
an artificial femoral head fixedly fitted on the connection portion of the cap body.

2. The femoral head prosthesis assembly as claimed in claim 1, wherein the cap body is formed with a central through hole, whereby a locating member can be passed through the through hole to extend into a top face of the protrusion portion of the femoral neck for locating the cap body.

3. The femoral head prosthesis assembly as claimed in claim 1, wherein the cap body is further formed with a locating hole, whereby an auxiliary locating member can be passed through the locating hole to extend into the femur.

4. The femoral head prosthesis assembly as claimed in claim 2, wherein the cap body is further formed with a locating hole, whereby an auxiliary locating member can be passed through the locating hole to extend into the femur.

5. The femoral head prosthesis assembly as claimed in claim 1, wherein the cap body further has at least one plate-like fixing extension portion, whereby at least one locating member can be passed through the fixing extension portion to penetrate into the femur.

6. The femoral head prosthesis assembly as claimed in claim 2, wherein the cap body further has at least one plate-like fixing extension portion, whereby at least one locating member can be passed through the fixing extension portion to penetrate into the femur.

7. The femoral head prosthesis assembly as claimed in claim 3, wherein the cap body further has at least one plate-like fixing extension portion, whereby at least one locating member can be passed through the fixing extension portion to penetrate into the femur.

8. The femoral head prosthesis assembly as claimed in claim 4, wherein the cap body further has at least one plate-like fixing extension portion, whereby at least one locating member can be passed through the fixing extension portion to penetrate into the femur.

9. The femoral head prosthesis assembly as claimed in claim 1, wherein the protrusion portion of the femoral neck is a hollow column with a straight periphery in parallel with the central line of femoral neck.

10. The femoral head prosthesis assembly as claimed in claim 2, wherein the protrusion portion of the femoral neck is a hollow column with a straight periphery in parallel with the central line of femoral neck.

11. The femoral head prosthesis assembly as claimed in claim 3, wherein the protrusion portion of the femoral neck is a hollow column with a straight periphery in parallel with the central line of femoral neck.

12. The femoral head prosthesis assembly as claimed in claim 4, wherein the protrusion portion of the femoral neck is a hollow column with a straight periphery in parallel with the central line of femoral neck.

13. The femoral head prosthesis assembly as claimed in claim 5, wherein the protrusion portion of the femoral neck is a hollow column with a straight periphery in parallel with the central line of femoral neck.

14. The femoral head prosthesis assembly as claimed in claim 6, wherein the protrusion portion of the femoral neck is a hollow column with a straight periphery in parallel with the central line of femoral neck.

15. The femoral head prosthesis assembly as claimed in claim 7, wherein the protrusion portion of the femoral neck is a hollow column with a straight periphery in parallel with the central line of femoral neck.

16. The femoral head prosthesis assembly as claimed in claim 8, wherein the protrusion portion of the femoral neck is a hollow column with a straight periphery in parallel with the central line of femoral neck.

17. The femoral head prosthesis assembly as claimed in claim 1, wherein a maximum junction cross section is taken between the root of the femoral neck and the femur, which is defined by a replacement loop, a bottom periphery of the cap body having a configuration in conformity to the replacement loop.

18. The femoral head prosthesis assembly as claimed in claim 2, wherein a maximum junction cross section is taken between the root of the femoral neck and the femur, which is defined by a replacement loop, a bottom periphery of the cap body having a configuration in conformity to the replacement loop.

19. The femoral head prosthesis assembly as claimed in claim 3, wherein a maximum junction cross section is taken between the root of the femoral neck and the femur, which is defined by a replacement loop, a bottom periphery of the cap body having a configuration in conformity to the replacement loop.

20. The femoral head prosthesis assembly as claimed in claim 4, wherein a maximum junction cross section is taken between the root of the femoral neck and the femur, which is defined by a replacement loop, a bottom periphery of the cap body having a configuration in conformity to the replacement loop.

21. The femoral head prosthesis assembly as claimed in claim 5, wherein a maximum junction cross section is taken between the root of the femoral neck and the femur, which is defined by a replacement loop, a bottom periphery of the cap body having a configuration in conformity to the replacement loop.

22. The femoral head prosthesis assembly as claimed in claim 6, wherein a maximum junction cross section is taken between the root of the femoral neck and the femur, which is defined by a replacement loop, a bottom periphery of the cap body having a configuration in conformity to the replacement loop.

23. The femoral head prosthesis assembly as claimed in claim 7, wherein a maximum junction cross section is taken between the root of the femoral neck and the femur, which is defined by a replacement loop, a bottom periphery of the cap body having a configuration in conformity to the replacement loop.

24. The femoral head prosthesis assembly as claimed in claim 9, wherein a maximum junction cross section is taken between the root of the femoral neck and the femur, which is defined by a replacement loop, a bottom periphery of the cap body having a configuration in conformity to the replacement loop.

25. The femoral head prosthesis assembly as claimed in claim 10, wherein a maximum junction cross section is taken between the root of the femoral neck and the femur, which is defined by a replacement loop, a bottom periphery of the cap body having a configuration in conformity to the replacement loop.

26. An operation instrument for femoral head replacement, comprising a femoral neck holder, which is a hollow casing with a configuration adapted to the surface configuration of the femoral neck, the femoral neck holder being sectioned at a middle portion of a hollow casing thereof to form a side opening, a guide tube being disposed on a top side of the femoral neck holder, the guide tube extending along the central line of the femoral neck for forming a central hole on the femoral neck, a transverse slot being formed on the middle portion of the femoral neck holder for indicating a cutting position.

27. The operation instrument for femoral head replacement as claimed in claim 26, wherein at least one locating hole is formed on the femoral neck holder, whereby a temporary retainer member can be passed through the locating hole to penetrate into the femur so as to fix the femoral neck holder thereon.

28. An operation instrument for femoral head replacement, comprising a shaper blade having a sleeve portion and a cutting blade portion, the cutting blade portion having a lower opening, a periphery of the opening being formed with a sharp blade section, the blade section having a dimension and a sectional configuration in conformity to those of a cutting loop composed of points of the periphery of the femoral neck, which points are spaced from a central line of the femoral neck by shortest distances, a guide member being disposed in a central hole of the femoral neck, the sleeve portion being fitted around the guide member to reciprocally move along the guide member.

29. The operation instrument for femoral head replacement as claimed in claim 28, wherein the cutting blade portion has a hollow column with a straight periphery in parallel with the central line of femoral neck.
